(19) United States
(12) Patent Application Publication
(10) Pub. No.: US 2008/0139961 A1
(43) Pub. Date: Jun. 12, 2008

(54) Osteomedullar Biopsy Trocar

(76) Inventors: Borhane Slama, Montfavet (FR); Hacene Zerazhi, Barbentane (FR)

Correspondence Address: ARNALL GOLDEN & GREGORY, LLP, 2800 ONE ATLANTIC CENTER, 1201 WEST PEACHTREE STREET, ATLANTA, GA, 30309, US

(21) Appl. No.: 11/632,915

(22) PCT Filed: Jul. 22, 2005
(86) PCT No.: PCT/FR2005/001905

§ 371 Date: Jan. 18, 2007

(30) Foreign Application Priority Data
Jul. 23, 2004 (FR) 0408216

(51) Int. Cl. A61B 10/02

(52) U.S. Cl. 600/567

(57) ABSTRACT

The invention concerns an osteomedullar biopsy trocar comprising an outer hollow tube provided with a sharpened distal end including an off-center orifice with a diameter smaller than the initial diameter of the tube, and an inner hollow tube located inside the first tube and whereof the distal end includes a sharpened crescent-shaped blade perpendicular to the axis of the trocar and arranged to be positioned laterally on the side opposite the off-center orifice so an to enable bone marrow to be introduced into the trocar during coring and the base of the core to be cut in the trocar axially rotating the inner tube and hence the blade.

### BACKGROUND OF THE INVENTION

This invention relates to a trocar for bone marrow biopsy.

In general it relates to the field of surgery and applies to the sampling of marrow from within patients' bones by coring for diagnostic purposes and therapeutic monitoring in the case of blood disorders, all bone marrow diseases, and blood diseases (lymphomas, leukemias, etc.).

At the present time hematologists and other specialists commonly carry out bone marrow biopsies using an instrument for sampling bone marrow comprising a hollow steel tube 1 and a removable punch 2 designed to be able to slide within the said tube (FIG. 1).

Hollow tube 1, which has a diameter of a few millimeters and a standard length of 130 millimeters, has a very sharp lower distal extremity 3 so that it is able to saw the bone circularly in order to penetrate the marrow located within so as to make a borehole providing access to the bone marrow, and then cut out a core of cylindrical shape 1.5 to 2 centimeters long and 3 to 4 millimeters in diameter from this in one single piece. The opposite extremity of hollow tube 1 is provided with a handle 4 of plastics material perpendicular to the tube located in the centre thereof which makes it possible to manipulate the said tube by exerting a pressure force and semicircular movements in order to cause the tube to penetrate the bone in order to saw the periosteum, cortical bone and the bone marrow circularly in the shape of a cylinder (coring).

Punch 2, which has a handle 5 at its proximal extremity, is first inserted within tube 1 (FIG. 2), as a result of which it can penetrate the surface of the skin and pass through the muscles until it comes into contact with the bone surface without invasion of the interior of the said tube. The punch is then withdrawn from the tube and the penetration movements in contact with the bone begin.

When a depth which is considered sufficient has been reached and the section of bone marrow lies within hollow tube 1, the practitioner has to detach the bone marrow core from the rest of the marrow, which is a hard, resistant and fibrous substance. In order to achieve this, large circular and pendular movements have to be made using handle 4 in an attempt to detach the core of bone marrow from the remainder of the marrow.

Unfortunately, this operation, which is essential for cutting off the base of the marrow core located in hollow tube 1 is random (non-systematic detachment), extremely painful and traumatic for the patient despite local anesthesia, and finally causes collateral damage to the periosteum, cortical bone and bone marrow.

This medical procedure, the result of which is not guaranteed, because in some cases the bone marrow core is not detached from the remainder of the bone marrow, worries a good many doctors and frightens patients.

The procedure is also long, which is a trial for the practitioner, and may exceed the time for which local anesthesia acts, so this must therefore be renewed periodically.

### DESCRIPTION OF THE PRIOR ART

A number of modifications to the conventional bone marrow biopsy trocar have been envisaged with a view to improving its conditions of use.

For example patent no. EP 1 175 866 (Worldwide Medical Technologies) describes an instrument for the extraction of bone marrow which comprises an internal cannula sliding in the outer hollow tube and includes a distal extremity equipped with longitudinal slots bounding flexible sectors. The said extremity of the inner cannula is designed to reduce in diameter when it is forced towards the extremity of the outer tube, in such a way as to grip the marrow in order to hold it when it is being extracted.

Patents Nos. WO 01 78 590 and 01 78 608 (Allegiance Corporation) disclose a bone marrow biopsy trocar incorporating an inner tube whose distal extremity comprises a cutting finger which acts through rotation.

Although they make the procedure easier, these instruments do not avoid the need to tear off cores by pendular or rotary movements.

The bone marrow biopsy trocar to which patent no. FR 2 610 508 (Orlovic) relates comprises a tube fitted with a cutting extremity which is caused to rotate by a motor, the extremity of an inner member housed within the said tube projecting from the said cutting extremity while passing through the soft tissues and being retracted while the bone tissue is being drilled, the said extremity of the inner member comprising a plurality of metal blades with folded ends sliding simultaneously within the rotating tube between an extended position in which the said blades form a projecting point at the extremity of the rotating tube as a result of being folded and a withdrawn position in which the said blades are housed within the tube and resiliently deformed in such a way that their originally folded extremities are caused to lie as an extension of the rest of the blade.

In the extended position the point formed by the blades perforates the soft tissues and prevents these from penetrating the tube, as is the case with the conventional punch. Once in contact with bone tissue the operator causes the blades to be drawn back into the withdrawn position in such a way that they are no longer in the way when the core is cut by the tube. Once the selected depth has been reached the operator causes the blades to move out into their extended position so that their extremities meet each other, passing through the core. The core is thus cut off at its base and it remains held within the trocar by the blades while the device is being withdrawn.

This is a complex and very cumbersome item of equipment which can only be considered for use in major operations performed in an operating theatre.

### SUMMARY OF THE INVENTION

The purpose of the device according to this invention is to overcome the disadvantages of known trocars while making it possible to construct an instrument which is easy to use and of affordable cost.

In particular it makes it possible to:
considerably reduce the pain inherent in the invasive procedure by eliminating circular and pendular movements,
ensure that the bone marrow core is sampled by cutting off its base and protecting it,
improve the quality of the bone marrow sampled by reducing microinjuries,
humanizing the procedure and making it practical and simple to perform.

These aims are accomplished through this invention comprising a bone marrow biopsy trocar incorporating a hollow outer tube provided with a sharp distal extremity comprising an eccentric orifice whose diameter is less than the initial diameter of the tube and also a hollow inner tube located within the first tube, a distal extremity of which comprises a sharpened crescent-shaped blade, perpendicular to the axis of the trocar and arranged so that it can be positioned laterally on the side opposite the eccentric orifice in such a way that it can penetrate the bone marrow in the trocar during coring and cut off the core at its base when the inner tube and therefore the blade is caused to rotate axially.

### BRIEF DESCRIPTION OF THE DRAWINGS

In the appended drawings, provided by way of a non-restrictive example of one embodiment according to this application:

FIG. 1, already mentioned, is a side elevation view of a existing bone marrow biopsy trocar with the punch separated from the outer tube,

FIG. 2, which has also been mentioned, illustrates the two elements of FIG. 1 assembled together,

FIG. 3 shows a bone marrow biopsy trocar according to the invention under the same conditions,

FIG. 4 is a magnified side view of the distal extremity of the outer tube of the trocar in FIG. 3,

FIG. 5 is an end view along the arrow F1 in FIG. 4,

FIG. 6 is a partially magnified side view of the inner tube of the trocar in FIG. 3,

FIG. 7 is an end view along the arrow F2 in FIG. 6,

FIG. 8 is a magnified axial cross-section of the extremity of the trocar in FIG. 3, with the inner tube in place in the outer tube,

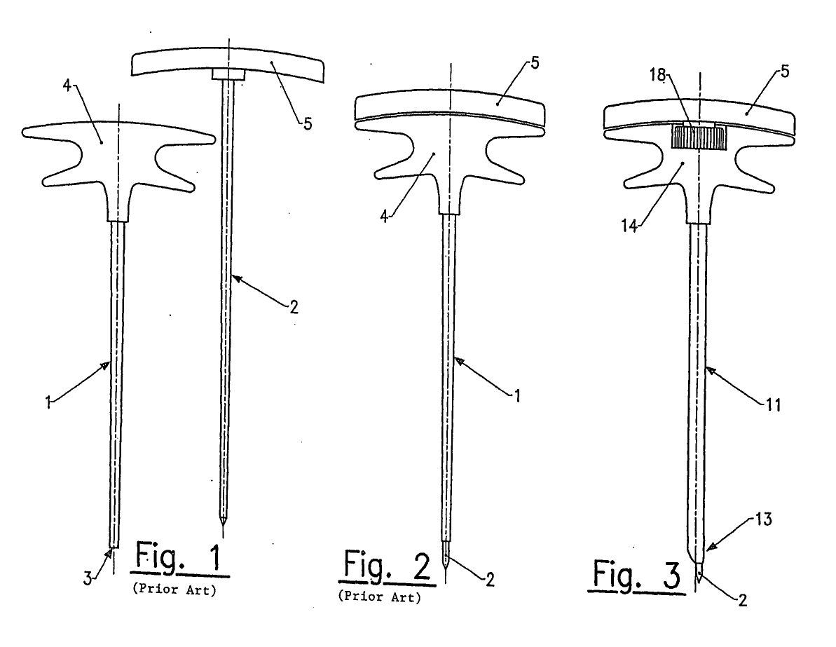

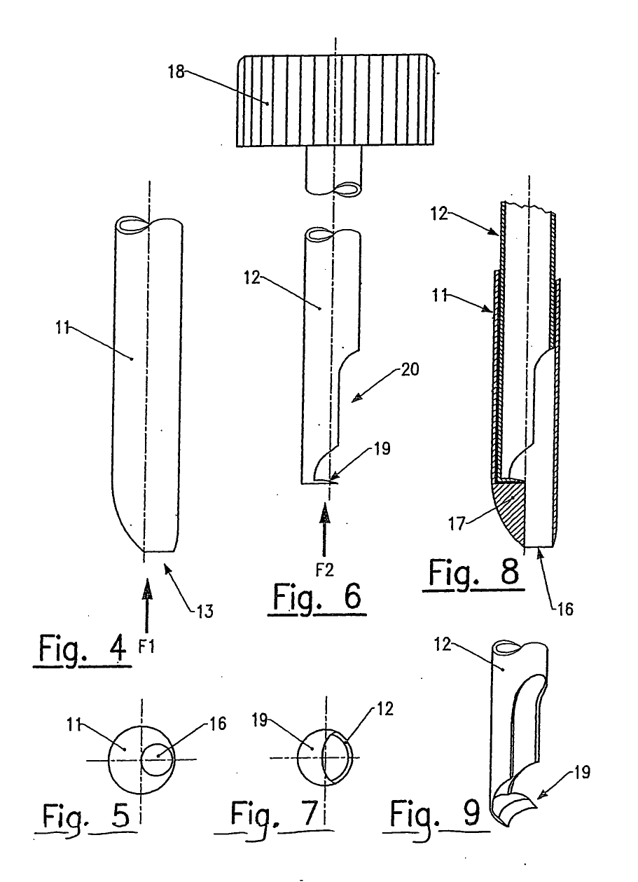

and FIG. 9 is an axonometric perspective view of the distal extremity of the inner tube showing the blade cutting the bone marrow core.

### DESCRIPTION OF THE PREFERRED EMBODIMENT

The device, FIGS. 3 to 9, comprises the assembly of a hollow outer tube 11 and a hollow inner tube 12 located within the first tube capable of receiving a movable punch 2 of the type currently in use to penetrate the surface of the skin and pass through the muscles until contact is made with the bone surface.

Hollow outer tube 11 comprises a proximal extremity fitted with a handle 14 through which the said trocar can be manipulated and a sharpened distal lower extremity 13 capable of penetrating a bone by boring and comprising an eccentric opening 16 whose diameter is less than the initial internal diameter of the outer tube. This extremity progressively widens over the outer half of the circumference of the tube over a height of several millimeters in such a way as to form a crescent-shaped base 17 comprising a planar surface perpendicular to the axis of the said tube within the tube.

Orifice 16 is preferably of circular shape.

The proximal extremity of hollow inner tube 12 is provided with an integral operating button or handle 18 through which axial rotary movement may be imparted to it.

The distal extremity of the said hollow inner tube comprises a sharpened crescent-shaped blade 19 perfectly matching base member 17 located at the lower extremity of outer tube 11 and a lateral opening 20 a few centimeters high diametrically opposite to the blade.

After axial rotation, blade 19 serves to support the bone marrow core in order to protect it and ensure that it is extracted.

Hollow inner tube 12 may be movable in order to assist cleaning and disinfection of the device, in a situation where the equipment is reusable.

The positioning of the various constituent elements provides the object of the invention with a maximum number of useful effects which have not hitherto been achieved by similar devices.

1. A bone marrow biopsy trocar intended for sampling of marrow within patients' bones by coring, comprising
an outer hollow tube provided with a sharpened distal extremity capable of penetrating a bone by boring and comprising an eccentric opening whose diameter is less than an initial internal diameter of the tube and a rotating hollow inner tube designed to be able to cut off and extract a core being sampled,
wherein the distal extremity of the hollow outer tube widens progressively over an outer half of a circumference of the tube to a height of a few millimeters in such a way as to constitute a crescent-shaped base within the tube comprising a planar surface perpendicular to an axis of the tube, and
wherein a distal extremity of the inner tube comprises a sharpened crescent-shaped blade perpendicular to an axis of the trocar and designed to be capable of being positioned laterally on a side opposite the eccentric opening in such a way that it is able to penetrate the bone marrow within the trocar during coring and also to cut off the core at its base as a result of the inner tube and therefore the blade being caused to rotate axially.

2. The bone marrow biopsy trocar according to claim 1, wherein the blade perfectly matches the base member located at the lower extremity of the outer tube.

3. The bone marrow biopsy trocar according to claim 1, wherein the opening is of circular shape.

4. The bone marrow biopsy trocar according to claim 1, wherein the distal extremity of the hollow inner tube comprises a lateral opening a few centimeters high diametrically opposite the blade.

5. The bone marrow biopsy trocar according to claim 1, wherein the blade is arranged in such a way that after rotation it acts as a support for the bone marrow core in order to protect it and ensure that it is extracted.

6. The bone marrow biopsy trocar according to claim 1, wherein the proximal extremity of the hollow inner tube is provided with an integral maneuvering button or handle through which axial rotary movement may be imparted to it.
